(19) United States
(12) Patent Application Publication
(10) Pub. No.: US 2014/0163350 A1
(43) Pub. Date: Jun. 12, 2014

(54) SILICON NANO-CRYSTAL BIOSENSOR AND METHOD OF FABRICATING THE SAME

(71) Applicant: Electronics and Telecommunications Research Institute, Daejeon (KR)

(72) Inventors: Chul Huh, Daejeon (KR); Sang Hyeob Kim, Daejeon (KR); Byoung-Jun Park, Iksan (KR); Eun Hye Jang, Sejong (KR); Myung-Ae Chung, Daejeon (KR)

(21) Appl. No.: 13/941,058

(22) Filed: Jul. 12, 2013

(30) Foreign Application Priority Data
Dec. 12, 2012 (KR) 10-2012-0144279

(51) Int. Cl. A61B 5/00; H01L 31/18

(52) U.S. Cl. CPC A61B 5/6867; H01L 31/18; B82Y 5/00
USPC 600/395; 438/24; 977/954

(57) ABSTRACT

A silicon nano-crystal biosensor includes a flexible substrate transformed depending on a shape of a body organ, a light emitting device disposed on the flexible substrate and emitting light, and a light detector opposite to the light emitting device on the flexible substrate. The light detector absorbs the emitted light. A length of the flexible substrate is substantially equal to or greater than a radius of curvature of the body organ.

### CROSS-REFERENCE TO RELATED APPLICATIONS

This U.S. non-provisional patent application claims priority under 35 U.S.C. §119 to Korean Patent Application No. 10-2012-0144279, filed on Dec. 12, 2012, the entirety of which is incorporated by reference herein.

### BACKGROUND

The inventive concept relates to silicon nano-crystal biosensors and methods of fabricating the same and, more particularly, to flexible silicon nano-crystal biosensors and methods of fabricating the same.

A biosensor includes a bioreceptor and a signal transducer to selectively sense a desired biomaterial. The bioreceptor may selectively react and be combined with a specific biomaterial. For example, the bioreceptor may be ferment, protein, a receptor, a cell, tissue, or deoxyribonucleic acid (DNA). The signal transducer may use electrochemistry, fluorescence, optics, a chromophilic property, or a piezoelectric property. Biosensors may be applied to various fields such as a medical field, an environmental field, analysis of infectious pathogens, an army, an industry, and a research field.

Generally, signal transducing methods used as a method of sensing the biomaterial may be classified into an electrochemical method and an optic method. The electrochemical method may sense and detect a weak signal outputted from the biomaterial in a sample. In the optic method, light may be irradiated to the biomaterial and then an optical signal outputted from the biomaterial may be detected to analyze the biomaterial.

An optical biosensor using the optic method may generally include a light source generating light and a light detector measuring the optical signal. A laser may be used as the light source, and a spectrometer may be used as the light detector for measuring the optical signal. The laser of the optical biosensor generally uses a compound semiconductor thin layer. However, it may be difficult to grow the compound semiconductor thin layer on a substrate and the growing process of the thin layer may use an expensive process gas. Additionally, since a general compound semiconductor thin layer is grown on a non-silicon based substrate, it may be difficult to integrate the compound semiconductor thin layer with a silicon electronic device for constituting a circuit of the biosensor.

### SUMMARY

Embodiments of the inventive concept may provide flexible silicon nano-crystal biosensors capable of reducing fabrication costs.

Embodiments of the inventive concept may also provide method of fabricating a flexible silicon nano-crystal biosensor capable of reducing fabrication costs.

In one aspect, a silicon nano-crystal biosensor may include: a flexible substrate transformed depending on a shape of a body organ; a light emitting device disposed on the flexible substrate and emitting light; and a light detector opposite to the light emitting device on the flexible substrate and absorbing the emitted light. A length of the flexible substrate may be substantially equal to or greater than a radius of curvature of the body organ.

In an embodiment, the flexible substrate may include a polymer substrate, a liquid crystal polymer substrate, a metal substrate, or a glass substrate.

In an embodiment, the light emitting device may include a hole-injection layer, a light emitting layer, an electron-injection layer, and a device electrode which are sequentially stacked on the flexible substrate.

In an embodiment, the light emitting layer may include silicon carbide (SiC) including a silicon nano-crystal.

In an embodiment, the silicon nano-crystal may have a size of about 1 nm to about 10 nm.

In an embodiment, the hole-injection layer and the electron-injection layer may include silicon carbide (SiC) or silicon-carbon nitride (SiCN).

In an embodiment, the light detector may include a hole doping layer, a light absorbing layer, an electron doping layer, and a detection electrode which are sequentially stacked on the flexible substrate.

In an embodiment, the light absorbing layer may include silicon carbide (SiC) including a silicon nano-crystal.

In an embodiment, the hole doping layer and the electron doping layer may include silicon carbide (SiC) or silicon-carbon nitride (SiCN).

In another aspect, a silicon nano-crystal biosensor may include: a measuring part having a through region, the measuring part formed of a flexible material; a light emitting part disposed at a side of the measuring part, the light emitting part emitting light to the measuring part; and a light detecting part disposed at another side of the measuring part, the light detecting part opposite to the light emitting part, and the light detecting part absorbing the light passing through the through region. An intensity of the light absorbed in the light detecting part may be varied according to a light absorption amount of biomaterials disposed in the through region.

In an embodiment, the measuring part may include a polymer material, a liquid crystal polymer material, a metal material, or a glass material.

In an embodiment, the light emitting part may include silicon carbide (SiC) including a silicon nano-crystal.

In an embodiment, the silicon nano-crystal may have a size of about 1 nm to about 10 nm.

In an embodiment, the light detecting part may include silicon carbide (SiC) including a silicon nano-crystal.

In still another aspect, a method of fabricating a silicon nano-crystal biosensor may include: forming a light emitting device and a light detector on a silicon substrate; separating the light emitting device and the light detector from the silicon substrate; and bonding the light emitting device and the light detector on a flexible substrate. The light emitting device and light detector may be spaced apart from each other on the flexible substrate.

In an embodiment, the silicon substrate may include a first silicon substrate and a second silicon substrate. In this case, forming the light emitting device and the light detector may include: sequentially forming a hole-injection layer, a light emitting layer, an electron-injection layer, and a device electrode on the first silicon substrate; and sequentially forming a hole doping layer, a light absorbing layer, an electron doping layer, and a detection electrode on the second silicon substrate.

In an embodiment, each of the light emitting layer and the light absorbing layer may be formed by a plasma enhanced chemical vapor deposition (PECVD) process using a silane (SiH₄) gas and a methane (CH₄) gas.

In an embodiment, each of the light emitting layer and the light absorbing layer may include silicon carbide (SiC) including a silicon nano-crystal.

In an embodiment, the light emitting layer and the light detector may be separated from the silicon substrate by a chemical etching process or a physical etching process.

### BRIEF DESCRIPTION OF THE DRAWINGS

The inventive concept will become more apparent in view of the attached drawings and accompanying detailed description.

FIGS. 1A and 1B are cross-sectional views illustrating a method of fabricating a silicon nano-crystal biosensor according to some embodiments of the inventive concept;

FIGS. 2A and 2B are cross-sectional views illustrating silicon nano-crystal biosensors according to some embodiments of the inventive concept;

FIG. 3 is a cross-sectional view illustrating a silicon nano-crystal biosensor according to other embodiments of the inventive concept; and

FIG. 4 is a graph illustrating a measurement result of a biomaterial measured using a silicon nano-crystal biosensor according to some embodiments of the inventive concept.

### DETAILED DESCRIPTION OF THE EMBODIMENTS

The inventive concept will now be described more fully hereinafter with reference to the accompanying drawings, in which exemplary embodiments of the inventive concept are shown. The advantages and features of the inventive concept and methods of achieving them will be apparent from the following exemplary embodiments that will be described in more detail with reference to the accompanying drawings. It should be noted, however, that the inventive concept is not limited to the following exemplary embodiments, and may be implemented in various forms. Accordingly, the exemplary embodiments are provided only to disclose the inventive concept and let those skilled in the art know the category of the inventive concept. In the drawings, embodiments of the inventive concept are not limited to the specific examples provided herein and are exaggerated for clarity.

The terminology used herein is for the purpose of describing particular embodiments only and is not intended to limit the invention. As used herein, the singular terms “a,” “an” and “the” are intended to include the plural forms as well, unless the context clearly indicates otherwise. As used herein, the term “and/or” includes any and all combinations of one or more of the associated listed items. It will be understood that when an element is referred to as being “connected” or “coupled” to another element, it may be directly connected or coupled to the other element or intervening elements may be present.

Similarly, it will be understood that when an element such as a layer, region or substrate is referred to as being “on” another element, it can be directly on the other element or intervening elements may be present. In contrast, the term “directly” means that there are no intervening elements. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

Additionally, the embodiment in the detailed description will be described with sectional views as ideal exemplary views of the inventive concept. Accordingly, shapes of the exemplary views may be modified according to manufacturing techniques and/or allowable errors. Therefore, the embodiments of the inventive concept are not limited to the specific shape illustrated in the exemplary views, but may include other shapes that may be created according to manufacturing processes. Areas exemplified in the drawings have general properties, and are used to illustrate specific shapes of elements. Thus, this should not be construed as limited to the scope of the inventive concept.

It will be also understood that although the terms first, second, third etc. may be used herein to describe various elements, these elements should not be limited by these terms. These terms are only used to distinguish one element from another element. Thus, a first element in some embodiments could be termed a second element in other embodiments without departing from the teachings of the present invention. Exemplary embodiments of aspects of the present inventive concept explained and illustrated herein include their complementary counterparts. The same reference numerals or the same reference designators denote the same elements throughout the specification.

Moreover, exemplary embodiments are described herein with reference to cross-sectional illustrations and/or plane illustrations that are idealized exemplary illustrations. Accordingly, variations from the shapes of the illustrations as a result, for example, of manufacturing techniques and/or tolerances, are to be expected. Thus, exemplary embodiments should not be construed as limited to the shapes of regions illustrated herein but are to include deviations in shapes that result, for example, from manufacturing. For example, an etching region illustrated as a rectangle will, typically, have rounded or curved features. Thus, the regions illustrated in the figures are schematic in nature and their shapes are not intended to illustrate the actual shape of a region of a device and are not intended to limit the scope of example embodiments.

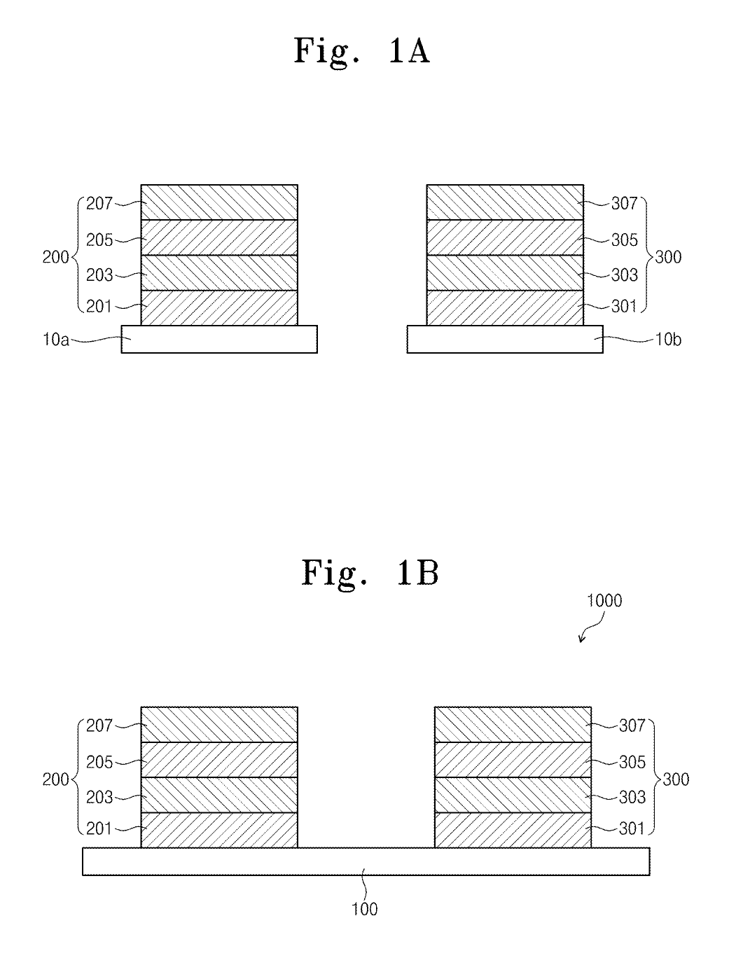

FIGS. 1A and 1B are cross-sectional views illustrating a method of fabricating a silicon nano-crystal biosensor according to some embodiments of the inventive concept.

Referring to FIG. 1A, a light emitting device 200 and a light detector 300 are formed on first and second silicon substrates 10a and 10b, respectively. The light emitting device 200 may include a hole-injection layer 201, a light emitting layer 203, an electron-injection layer 205, and a device electrode 207 which are sequentially stacked on the first silicon substrate 10a. The hole-injection layer 201 may be formed of a p-type silicon carbide (SiC) layer or a p-type silicon-carbon nitride (SiCN) layer. The electron-injection layer 205 may be formed of an n-type silicon carbide (SiC) layer or an n-type silicon-carbon nitride (SiCN) layer. The light emitting layer 203 may be formed by depositing a silicon carbide layer including a silicon nano-crystal. In more detail, the light emitting layer 203 may be formed by a plasma enhanced chemical vapor deposition (PECVD) process using a silane (SiH₄) gas and a methane (CH₄) gas. In other words, the silane gas may react with the methane gas to form the light emitting layer 203 in the PECVD process. The device electrode 207 may be formed by depositing an indium-tin oxide (ITO) layer, a tin oxide (SnO₂) layer, an indium oxide (In₂O₃) layer, a cadmium-thin oxide (Cd₂SnO₄) layer, or a zinc oxide (ZnO) layer.

The light detector 300 may include a hole doping layer 301, a light absorbing layer 303, an electron doping layer 305, and a detection electrode 307 which are sequentially stacked on the second silicon substrate 10b. The hole doping layer 301 may be formed of a p-type silicon carbide (SiC) layer or a p-type silicon-carbon nitride (SiCN) layer. The electron doping layer 305 may be formed of an n-type silicon carbide (SiC) layer or an n-type silicon-carbon nitride (SiCN) layer. The light absorbing layer 303 may be formed by depositing a silicon carbide layer including a silicon nano-crystal. In more detail, a silane (SiH₄) gas may react with a methane (CH₄) gas to form the light absorbing layer 303 in a PECVD process. The detection electrode 307 may be formed by depositing an indium-tin oxide (ITO) layer, a tin oxide (SnO₂) layer, an indium oxide (In₂O₃) layer, a cadmium-thin oxide (Cd₂SnO₄) layer, or a zinc oxide (ZnO) layer.

Alternatively, a p-type silicon layer, a silicon nano-crystal layer, an n-type silicon layer, and a transparent electrode layer may be sequentially deposited on one silicon substrate, and then the deposited layers may be successively patterned to form the light emitting device 200 and the light detector 300 on the one silicon substrate.

Referring to FIG. 1B, the light emitting device 200 and the light detector 300 may be separated from the silicon substrates 10a and 10b, and then the separated light emitting device 200 and the separated light detector 300 may be bonded to a flexible substrate 100, thereby forming a silicon nano-crystal biosensor 1000.

The light emitting device 200 and the light detector 300 may be separated from the silicon substrates 10a and 10b by a physical etching process or a chemical etching process. The separated light emitting device 200 and light detector 300 may be bonded to the flexible substrate 100 by a physical bonding process (e.g., a pressing process using heat) or a chemical bonding process. The flexible substrate 100 may have flexibility. Additionally, the flexible substrate 100 may include a transparent material. The flexible substrate may be, for example, a polymer substrate, a liquid crystal polymer substrate, a metal substrate, or a glass substrate.

A surface of the silicon nano-crystal biosensor 1000 may be capped with a polymer material having an adhesive property with respect to a body.

The light emitting device 200 and the light detector 300 including the silicon carbide (SiC) may be formed on the silicon substrates 10a and 10b by using the PECVD process using low-cost silane (SiH₄) and low-cost methane (CH₄) gases. Thus, the silicon nano-crystal biosensor 1000 does not require an additional optic system. As a result, a fabrication cost of the silicon nano-crystal biosensor 1000 may be reduced.

Additionally, the light emitting device 200 and the light detector 300 may be bonded to the flexible substrate 100 to form the silicon nano-crystal biosensor 1000. Thus, due to the flexible substrate 100, a shape of the silicon nano-crystal biosensor 1000 may be easily transformed to be suitable to any one of various shapes of a body organ (e.g., an internal organ such as a blood vessel or the heart) and then may be bonded or transplanted to an outer wall thereof.

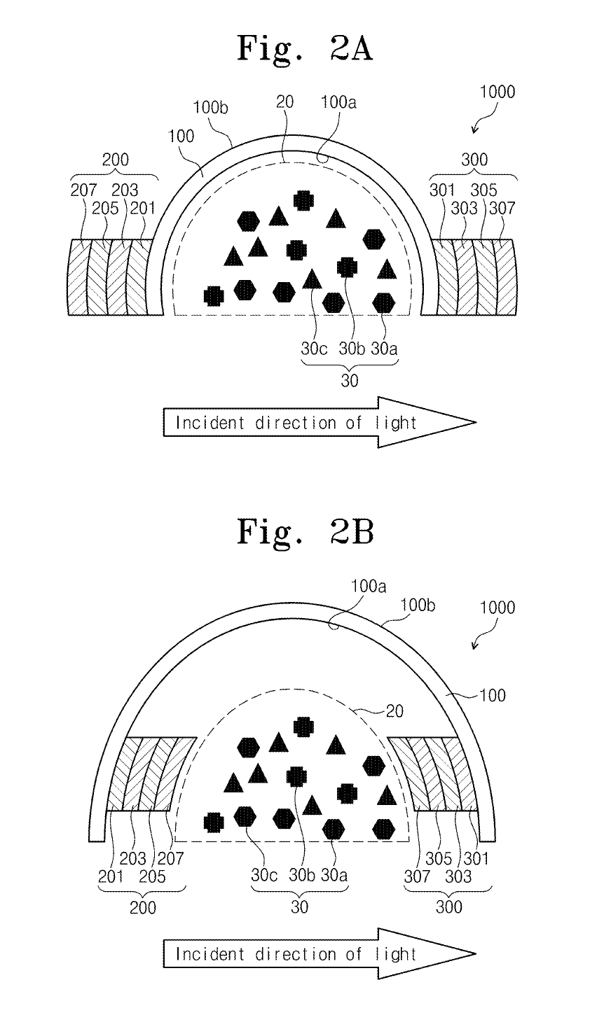

FIGS. 2A and 2B are cross-sectional views illustrating silicon nano-crystal biosensors according to some embodiments of the inventive concept.

Referring to FIGS. 2A and 2B, a silicon nano-crystal biosensor 1000 may include a flexible substrate 100, a light emitting device 200, and an light detector 300. The light emitting device 200 and the light detector 300 may be formed on the flexible substrate 100. A surface of the silicon nano-crystal biosensor 1000 may be capped with a polymer material (not shown) having an adhesive property with respect to a body.

The flexible substrate 100 may have flexibility. Thus, if the flexible substrate 100 is bent, the flexible substrate 100 may have a first surface 100a and a second surface 100b which correspond to an inner surface and an outer surface, respectively. In other words, the first and second surfaces 100a and 100b may be curved surfaces.

As illustrated in FIG. 2A, the first surface 100a of the flexible substrate 100 may define a region in which a body organ 20 (e.g., an internal organ such as a blood vessel or the heart) including a body fluid is disposed. The light emitting device 200 and the light detector 300 may be disposed to be opposite to each other on the second surface 100b of the flexible substrate 100. Since light emitted from the light emitting device 200 travels to the light detector 300 through the body organ 20, the flexible substrate 100 may be formed of a transparent material.

Alternatively, as illustrated in FIG. 2B, the light emitting device 200 and the light detector 300 may be disposed to be opposite to each other on the first surface 100a of the flexible substrate 100, and the body organ 20 including the body fluid may be disposed to be adjacent to the first surface 100a of the flexible substrate 100. In other words, the first surface 100a may surround the body organ 20. Thus, the light emitting device 200 and the light detector 300 may be in contact with the body organ 20. In this case, the flexible substrate 100 may be formed of a transparent or opaque material. For example, the flexible substrate 100 may be a polymer substrate, a liquid crystal polymer substrate, a metal substrate, or a glass substrate.

Biomaterials 30 may be included in the body fluid (e.g., blood, urine, and/or tears). Thus, a length of the flexible substrate 100 may be substantially equal to or greater than a radius of curvature of the body organ 20.

The light emitting device 200 may include a hole-injection layer 201, a light emitting layer 203, an electron-injection layer 205, and a device electrode 207.

The hole-injection layer 201 may include a p-type silicon material, and the electron-injection layer 205 may include an n-type silicon material. For example, the hole-injection layer 201 and the electron-injection layer 205 may include silicon carbide (SiC) or silicon-carbon nitride (SiCN). Each of the hole-injection layer 201 and the electron-injection layer 205 may have a thickness of about 1 nm or more. The hole-injection layer 201 may supply holes to the light emitting layer 203, and the electron-injection layer 205 may supply electrons to the light emitting layer 203.

The light emitting layer 203 may be disposed between the hole-injection layer 201 and the electron-injection layer 205. The light emitting layer 203 may be formed of silicon carbide (SiC) including a silicon nano-crystal. The silicon nano-crystal may have a size of about 1 nm to about 10 nm. The light emitting layer 203 may have a thickness of about 1 nm or more. The holes supplied from the hole-injection layer 201 may be combined with the electrons supplied from the electron-injection layer 205 in the light emitting layer 203, such that the light emitting layer 203 may generate light.

The device electrode 207 may be a transparent electrode. The device electrode 207 may include indium-tin oxide (ITO), tin oxide (SnO₂), indium oxide (In₂O₃), cadmium-thin oxide (Cd₂SnO₄), or zinc oxide (ZnO).

The light detector 300 may include a hole doping layer 301, a light absorbing layer 303, an electron doping layer 305, and a detection electrode 307.

The hole doping layer 301 may include a p-type silicon material, and the electron doping layer 305 may include an n-type silicon material. For example, the hole doping layer 301 and the electron doping layer 305 may include silicon carbide (SiC) or silicon-carbon nitride (SiCN). Each of the hole and electron doping layers 301 and 305 may have a thickness of about 1 nm or more. The hole doping layer 301 may receive holes from the light absorbing layer 303, and the electron doping layer 305 may receive electrons from the light absorbing layer 203.

The light absorbing layer 303 may be disposed between the hole and electron doping layers 301 and 305. The light absorbing layer 303 may be formed of silicon carbide (SiC) including a silicon nano-crystal. The light absorbing layer 303 may have a thickness of about 1 nm or more. The light absorbing may absorb the light emitted from the light emitting device 200.

The detection electrode 307 may be a transparent electrode. The detection electrode 307 may include indium-tin oxide (ITO), tin oxide (SnO₂), indium oxide (In₂O₃), cadmium-thin oxide (Cd₂SnO₄), or zinc oxide (ZnO).

A measurement method of the silicon nano-crystal biosensor 1000 will be described hereinafter. A positive voltage is applied to the hole-injection layer 201 of the light emitting device 200, and a negative voltage is applied to the electron-injection layer 205 of the light emitting device 200. A negative voltage is applied to the hole doping layer 301 of the light detector 300, and a positive voltage is applied to the electron doping layer 305. Thus, the light generated from the light emitting device 200 is incident on the body organ 20, and then the light detector 300 absorbs the incident light on the body organ 20. The intensity of the light absorbed into the light detector 300 may be varied depending on a concentration of the biomaterials 30. The concentration of the biomaterials 30 in the body fluid may be detected using this principle.

The biomaterials 30 may include different kinds of biomaterials 30a, 30b, and 30c. In this case, each kind of biomaterial 30a, 30b, or 30c may have a proper light absorption wavelength. Thus, the light emitting device 200 may be formed to generate light having the same wavelength as the proper light absorption wavelength of a detection target biomaterial, such that a high sensitive silicon nano-crystal biosensor 1000 may be realized.

The silicon nano-crystal biosensor 1000 uses the flexible substrate 100 in order to detect the biomaterials 30 included in the body fluid of the body organ 20 having a shape. Due to the flexibility of the flexible substrate, the silicon nano-crystal biosensor 1000 may be bonded to the outer wall of the body organ 20. As a result, the biomaterials 30 in the body fluid of the body organ 20 may be detected.

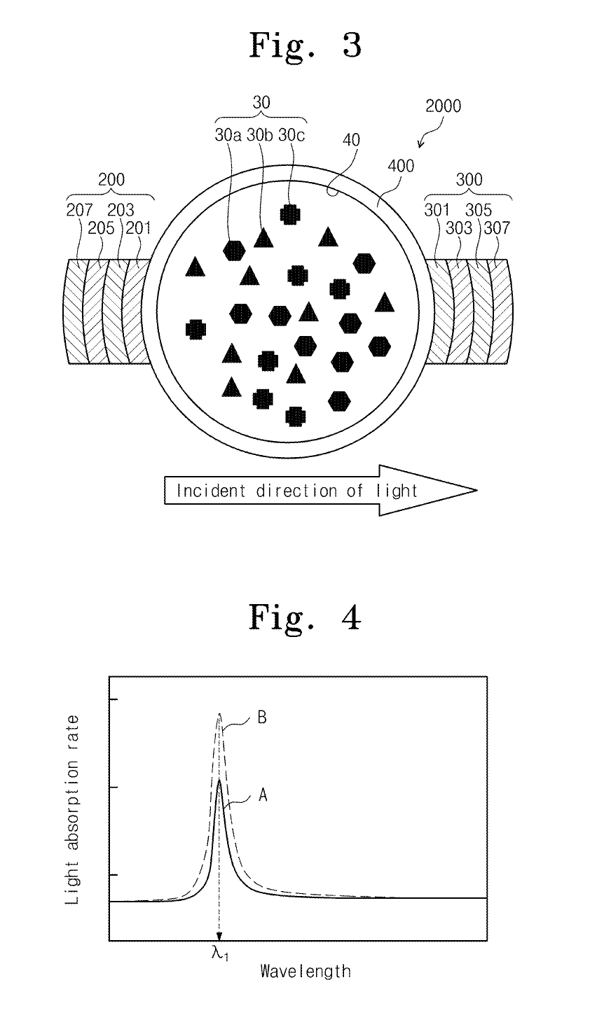

FIG. 3 is a cross-sectional view illustrating a silicon nano-crystal biosensor according to other embodiments of the inventive concept.

In the present embodiment of FIG. 3, the same elements as described in aforementioned embodiment will be indicated by the same reference numerals or the same reference designators, and the descriptions to the same elements will be omitted or mentioned briefly.

Referring to FIG. 3, a silicon nano-crystal biosensor 2000 may include a measuring part 400 having a through region 40, a light emitting part 200 disposed at a side of the measuring part 400, and a light detecting part 300 disposed at another side of the measuring part 400 and opposite to the light emitting part 200. A surface of the silicon nano-crystal biosensor 2000 may be capped with a polymer material having an adhesive property with respect to a body.

The measuring part 400 is formed of a flexible material. The measuring part 400 may include a polymer material, a liquid crystal polymer material, a metal material, and/or a glass material. If the light emitting part 200 and the light detecting part 300 are disposed outside the measuring part 400 as illustrated in FIG. 3, the measuring part 100 may be formed of a transparent material since light emitted from the light emitting part 200 is transmitted through the measuring part 400.

Alternatively, if the light emitting part 200 and the light detecting part 300 are disposed to face each other inside the measuring part 400, the measuring part 400 may be formed of a transparent or opaque material.

A blood vessel may be disposed in the through region 40. In an embodiment, the blood vessel may be an artificial vessel. The artificial vessel may be inserted into the through region 40 of the silicon nano-crystal biosensor 2000, and then the inserted artificial vessel may be transplanted to a blood vessel of the human body. Thus, the concentration of the target biomaterials may be detected from various kinds of biomaterials 30a, 30b, and 30c.

The light emitting part 200 and the light detecting part 300 may include silicon carbide (SiC) having a silicon nano-crystal. The silicon nano-crystal may have a size of about 1 nm to about 10 nm.

FIG. 4 is a graph illustrating a measurement result of a biomaterial measured using a silicon nano-crystal biosensor according to some embodiments of the inventive concept.

Referring to FIG. 4, a reference designator ‘A’ denotes a low-concentration body fluid, and a reference designator ‘B’ denotes a high-concentration body fluid. As illustrated in FIG. 4, a light absorption rate of the high-concentration body fluid B is greater than a light absorption rate of the low-concentration body fluid A with respect to a specific wavelength 4 such that the amount of the biomaterials in the body fluid B is greater than the amount of the biomaterials in the body fluid A.

According to embodiments of the inventive concept, the light emitting device and the light detector of the silicon nano-crystal biosensor include the silicon carbide (SiC) having the silicon nano-crystal. The silicon carbide (SiC) may be formed on the silicon substrate by the PECVD using the low-cost silane (SiH₄) and the low-cost methane (CH₄) gases. Thus, the fabrication cost of the silicon nano-crystal biosensor may be reduced or minimized.

Additionally, the silicon nano-crystal biosensor includes the flexible substrate which can be transformed to be suitable to the shape of the body organ. Thus, the silicon nano-crystal biosensor may be easily and suitably bonded or transplanted to the body organ, such that it may detect the biomaterials existing in the body fluid of the body organ.

While the inventive concept has been described with reference to example embodiments, it will be apparent to those skilled in the art that various changes and modifications may be made without departing from the spirit and scope of the inventive concept. Therefore, it should be understood that the above embodiments are not limiting, but illustrative. Thus, the scope of the inventive concept is to be determined by the broadest permissible interpretation of the following claims and their equivalents, and shall not be restricted or limited by the foregoing description.

What is claimed is:

1. A silicon nano-crystal biosensor comprising:
a flexible substrate transformed depending on a shape of a body organ;
a light emitting device disposed on the flexible substrate and emitting light; and
a light detector opposite to the light emitting device on the flexible substrate, the light detector absorbing the emitted light,
wherein a length of the flexible substrate is substantially equal to or greater than a radius of curvature of the body organ.

2. The silicon nano-crystal biosensor of claim 1, wherein the flexible substrate includes a polymer substrate, a liquid crystal polymer substrate, a metal substrate, or a glass substrate.

3. The silicon nano-crystal biosensor of claim 1, wherein the light emitting device includes a hole-injection layer, a light emitting layer, an electron-injection layer, and a device electrode which are sequentially stacked on the flexible substrate.

4. The silicon nano-crystal biosensor of claim 3, wherein the light emitting layer includes silicon carbide (SiC) including a silicon nano-crystal.

5. The silicon nano-crystal biosensor of claim 4, wherein the silicon nano-crystal has a size of about 1 nm to about 10 nm.

6. The silicon nano-crystal biosensor of claim 3, wherein the hole-injection layer and the electron-injection layer include silicon carbide (SiC) or silicon-carbon nitride (SiCN).

7. The silicon nano-crystal biosensor of claim 1, wherein the light detector includes a hole doping layer, a light absorbing layer, an electron doping layer, and a detection electrode which are sequentially stacked on the flexible substrate.

8. The silicon nano-crystal biosensor of claim 7, wherein the light absorbing layer includes silicon carbide (SiC) including a silicon nano-crystal.

9. The silicon nano-crystal biosensor of claim 7, wherein the hole doping layer and the electron doping layer include silicon carbide (SiC) or silicon-carbon nitride (SiCN).

10. A silicon nano-crystal biosensor comprising:
a measuring part having a through region, the measuring part formed of a flexible material;
a light emitting part disposed at a side of the measuring part, the light emitting part emitting light to the measuring part; and
a light detecting part disposed at another side of the measuring part, the light detecting part opposite to the light emitting part, and the light detecting part absorbing the light passing through the through region,
wherein an intensity of the light absorbed in the light detecting part is varied according to a light absorption amount of biomaterials disposed in the through region.

11. The silicon nano-crystal biosensor of claim 10, wherein the measuring part includes a polymer material, a liquid crystal polymer material, a metal material, or a glass material.

12. The silicon nano-crystal biosensor of claim 10, wherein the light emitting part includes silicon carbide (SiC) including a silicon nano-crystal.

13. The silicon nano-crystal biosensor of claim 12, wherein the silicon nano-crystal has a size of about 1 nm to about 10 nm.

14. The silicon nano-crystal biosensor of claim 10, wherein the light detecting part includes silicon carbide (SiC) including a silicon nano-crystal.

15. A method of fabricating a silicon nano-crystal biosensor, the method comprising:
forming a light emitting device and a light detector on a silicon substrate;
separating the light emitting device and the light detector from the silicon substrate; and
bonding the light emitting device and the light detector on a flexible substrate, the light emitting device and light detector spaced apart from each other on the flexible substrate.

16. The method of claim 15, wherein the silicon substrate includes a first silicon substrate and a second silicon substrate; and
wherein forming the light emitting device and the light detector comprises:
sequentially forming a hole-injection layer, a light emitting layer, an electron-injection layer, and a device electrode on the first silicon substrate; and
sequentially forming a hole doping layer, a light absorbing layer, an electron doping layer, and a detection electrode on the second silicon substrate.

17. The method of claim 16, wherein each of the light emitting layer and the light absorbing layer is formed by a plasma enhanced chemical vapor deposition (PECVD) process using a silane (SiH₄) gas and a methane (CH₄) gas.

18. The method of claim 17, wherein each of the light emitting layer and the light absorbing layer include silicon carbide (SiC) including a silicon nano-crystal.

19. The method of claim 15, wherein the light emitting layer and the light detector are separated from the silicon substrate by a chemical etching process or a physical etching process.
